(19) United States
(12) Patent Application Publication
(10) Pub. No.: US 2011/0212508 A1
(43) Pub. Date: Sep. 1, 2011

(54) Novel Synthetic Expression Vehicle

(76) Inventors: Laxmi Srinivas Rao, Maharashtra (IN); Shrikant Mishra, Maharashtra (IN); Milind Niphadkar, Maharashtra (IN); Priti Thakur, Maharashtra (IN); Genevieve Nazareth, Maharashtra (IN)

(21) Appl. No.: 13/123,960

(22) PCT Filed: Oct. 28, 2009
(86) PCT No.: PCT/IN2009/000610

§ 371 Date: Apr. 13, 2011

(30) Foreign Application Priority Data
Nov. 18, 2008 (IN) 2426/MUM/2008

(51) Int. Cl. C12N 1/21; C12N 15/63; C07H 21/04

(52) U.S. Cl. 435/252.33; 435/320.1; 536/24.1

(57) ABSTRACT

An expression vehicle comprising an isolated nucleic acid as shown in Seq ID No. 1 comprising of a synthetic hybrid promoter wherein the hybrid promoter comprises of an inducible arabinose promoter derived from E. coli and a synthetic stretch of 8-35 nucleotides from pHO regulon introduced in the region between −55 to −10 and ribosome binding site from the transcription initiation site.

### RELATED APPLICATION

This application claims the benefit of Indian Provisional application number 2426/MUM/2008 filed on Nov. 18, 2008.

### TECHNICAL FIELD

The present invention relates to a protein expression vector and uses thereof. More particularly, it relates to a protein expression vector with a synthetic hybrid promoter which can express a gene encoding a target protein in various E. coli strains to produce the said protein.

### BACKGROUND AND PRIOR ART

A variety of expression vectors have heretofore been developed for use in the production of recombinant proteins, in particular, for the expression systems utilizing microorganisms such as Escherichia coli and yeasts as hosts. In the systems utilizing Escherichia coli as the host, expressing capacity can be enhanced by using a potent promoter derived from Escherichia coli. Many expression platforms in E. coli and related bacteria have incorporated only a limited set of bacterial promoters. The most widely used bacterial promoters have included the lactose (lac) (Yanisch-Perron et al., 1985, Gene 33:103-109), and the tryptophan (trp) (Goeddel et al., 1980, Nature (London) 287:411-416) promoters, and the hybrid promoters derived from these two (tac and trc) (Brosius, 1984, Gene 27:161-172; and Amanna and Brosius, 1985, Gene 40:183-190). Other commonly used bacterial promoters include the phage lambda promoters pL and pR (Elvin et al., 1990, Gene 37:123-126), the phage T7 promoter (Tabor and Richardson, 1998, Proc. Natl. Acad. Sci. USA. 82:1074-1078), and the alkaline phosphatase promoter (pho) (Chang et al.,1986, Gene 44:121-125). Every promoter has its own characteristic response and an ideal promoter is one which offers additional features and often expresses the recombinant protein in relatively higher yields as compared to promoters used in the existing vector platforms. It is preferable for the promoter to tightly regulate gene expression during culture propagation (as many recombinant proteins can be toxic to the expression host). In contrast, when gene expression is desired, the promoter must be easily controlled and a high expression level is often preferred. Also, the inducer initiating the gene expression should be nontoxic, easily accessible, cheap and easily disposable post fermentation run. Presently two major platforms are utilised for high expression level of heterologous protein in prokaryotes. One is the pET series of expression vectors wherein the expression is induced from the strong T7 lac promoter and the other is pL and pR series of temperature sensitive expression platforms. The BL21 E. coli expression strain gives improved mRNA stability further increasing protein yields. Another expression system wherein titrable expression of protein is tightly controlled through the presence of specific carbon sources such as glucose, glycerol, and arabinose.

Commercial vectors having the araB bacterial promoter of the Enterobacteriaceae family has proven to be particularly advantageous for providing tightly repressed gene expression in the absence of the inducer arabinose and highly derepressed gene expression in the presence of the inducer arabinose. U.S. Pat. No. 5,028,530 specifically describes a replicable expression vehicle comprising the sequence of araB promoter from a member of the Enterobacteriaceae family; and gene of interest encoding heterologous protein operably linked to said promoter such that the gene is expressed in a given araC+ host for said vehicle by induction of said araB promoter. Marc Better, 1999, in Gene Expression Systems: Using Nature for the Art of Expression, Academic Press, New York, pp. 105, has disclosed the inherent advantages of araBAD promoter as follows: i) genes under ara control are tightly repressed in the absence of inducer, ii) upon induction, the resulting protein can be produced 1000 fold or more over the uninduced level, iii) arabinose is widely available and relatively inexpensive, iv) very little arabinose is required for full induction, v) processes using araB promoter are easily scalable, vi) in processes using araB promoter, expression yield is high, vii) It is a versatile system which can function in a variety of E. coli strains as well as in other bacterial species, viii) works for both secretory proteins as well as intracellular proteins, and most importantly ix) the control elements for the ara system are conveniently contained within about 300 by of DNA. Thus the two features of the ara system which has made it particularly well-suited for expression of recombinant proteins in E. coli are: i) it is simple to exploit because the control element of the araB promoter are conveniently contained within an approximately 300 base pair regulatory region and only a functional coding sequence for the araC gene is additionally needed. ii) regulation of the system has proven to be particularly tight, i.e., the ratio of the amount of the product in the induced state (with arabinose) relative to that in the repressed state (without arabinose) from the araB promoter on multicopy expression plasmids is relatively high, most frequently in the range from >200-75,000 (Better et al., 1999, in Gene Expression Systems: Using Nature for the Art of Expression, Academic Press, New York, pp. 95-107).

The following disclosures relate to the use of the araB promoter for the expression of polypeptides in bacteria. Johnston et al., 1985, Gene 34:137-145, disclose the cloning of MI3 gene II in pINGI plasmid placed under the control of the inducible araB promoter of Salmonella typhimurium and expression of said gene II protein to a level of almost 15% of the total protein in Escherichia coli cells. Restriction sites were introduced into the coding region of araB gene so that a gene fusion or a multigene transcription unit could be expressed under arabinose control. Jacobs et al., 1989, Gene 83:95-103 disclose a synthetic gene encoding human metallothionein-II (HMT) cloned into the specially constructed high-copy number expression vector, pUA7, and expressed in Escherichia coli. The plasmid construct includes the promoter/operator and regulatory sequences of the Salmonella typhimurium ara operon and part of the 5′-coding and all of the 3′-noncoding regions of the E. coli fusion protein. Upon induction with arabinose, the resulting fusion protein produced 75000-fold over uninduced cells, with a relatively stable mRNA (T1/2 of 8.3 minutes) and a completely stable protein. Cells producing the fusion protein bioaccumulated heavy metals 66 fold over nonproducing cells. This system was used to express an active heterologous protein that previously had been somewhat toxic and unstable in E. coli. Cagnon et al., 1991, Protein Eng. 4:843-847, discloses a set of expression vectors that contain the ara expression system from pINGI in the vector pKK233.2 along with a number of other optional features. In this series of expression vectors, the promoter/operator region of araB was followed by a polylinker region for convenient gene cloning. Other features are f1origin of replication (in plus or minus orientation) and a promoter^{up} mutation that enhances the level of expression from these vectors. The mutated araB promoter incorporated changes in the −10 region that made the promoter match more closely a consensus E. coli promoter. The promoter mutations resulted in higher level nearly 2 fold of inducible expression for a marker gene, however, the uninduced expression level increased as well. Several recombinant proteins were expressed from this family of ara expression vectors including the full length Tat protein from the HIV virus (Armenguad et al., 1991, FEBS Lett. 282: 157-160) and the bacterial proteins:β-galactosidase (Cagnon et al., 1991), the Streptoalloteichus hindustanus bleomycin-binding protein (Cagnon et al., 1991), and the cholera toxin subunit B (Slos et al., 1994, Protein Express. Purif. 5:518-526). The cholera toxin subunit B(CT-B) was linked to the OmpA signal sequence and expressed as a secreted protein. CT-B accumulated to approximately 60% of the total periplasmic protein and CT-B was produced at about 1 g/L at pilot scale. Perez-Perez and Gutierrez, 1995, Gene 158: 141-142, described arabinose inducible genetic elements from the Salmonella typhimurium arabinose operon inserted into pACYC184. The resultant plasmid, pAR3, was compatible with ColEI-derived plasmids and allowed efficient expression of recombinant genes upon induction with arabinose. Guzman et al., 1995, J Bacteria 177:4121-4130, described a series of araB expression vectors that incorporate various selectable markers and multicloning sites. This series of vectors were studied extensively for the expression of native E. coli proteins. Guzman et al. (1995) also presented evidence that the araB system can be used to achieve very low levels of uninduced expression, obtain moderately high levels of expression in the presence of inducer, and modulate expression over a wide range of inducer concentrations. The extent of arabinose induction can be regulated by the amount of inducer added to the culture. U.S. Pat. No. 6,803,210 discloses improved methods for the expression of recombinant protein products under the transcriptional control of an inducible promoter, such as an araB promoter, in bacterial host cells that are deficient in one or more of the active transport systems for an inducer of an inducible promoter, such as arabinose for an araB promoter, and contain an expression vector encoding a recombinant polypeptide under the transcriptional control of the inducible promoter, such as an araB promoter. The references cited above indicate that the araB expression system is useful for the controlled expression of recombinant proteins in bacterial systems.

In the above prior art, the development of expression vectors has been attempted primarily along two approaches, namely an attempt to simplify the purification of expressed recombinant proteins by either use of a secretory signal peptide or use of histidine tag to enhance the purification efficiency and the second major approach aiming at enhancing the expression levels.

Under the above-described circumstances, it has been desired to develop an expression vector that can give high expression of recombinant proteins in prokaryotic hosts, preferably E. coli wherein the expression is controlled by strong inducible synthetic hybrid promoter with transcriptional activator placed upstream of the start codon ATG for better transcription initiation. Also, it is a contention to improve the transcription efficiency by base substitutions in said vector by elimination of out of frame start and stop codons.

### OBJECT OF INVENTION

Accordingly, the primary object of the present invention is to provide an expression vector with a synthetic hybrid promoter which can express a gene encoding a target protein in E. coli strains to produce said protein. Another object of the invention is use of the said expression vector for high level expression of several heterologous proteins. Yet another object of the present invention is construction of an expression vehicle with strong induction from hybrid ara and pho regulons of Escherichia coli in this promoter. Another object of the invention is to provide a synthetic promoter that is more efficient than araBAD promoter in induction of protein expression. Another object of the invention is to obtain high protein expression in short time post induction and consistent expression levels thereafter till the end of fermentation run. Still another object of invention is introduction of a series of pHO box sequence variants of variable size between −55 to −10 base pairs and ribosome binding site from the transcription initiation site in the expression vehicle.

### SUMMARY OF THE INVENTION

The present invention provides an expression vector with a synthetic hybrid promoter which can express a gene encoding a target protein in various E. coli strains to produce said protein. The expression vector provided herein is primarily used for high level expression of said target proteins expressed as inclusion bodies in E. coli, amounting to atleast 20% expression of the total protein. The present invention targets to obtain high protein expression in short time post induction and consistent expression levels thereafter till the end of fermentation run. The present invention introduces of a series of pHO box sequence variants of variable size between −55 to −10 base pairs and ribosome binding site from the transcription initiation site in the expression vehicle.

### BRIEF DESCRIPTION OF ACCOMPANYING DRAWINGS

The manner in which the objects and advantages of the invention may be obtained will appear more fully from the detailed description and accompanying drawings, which are as follows:

FIG. 1: Map of pRA-LacZ vector
RA promoter region: bases 2-250; Initiation ATG: Bases 319-321;
Polyhistidine tag: bases 331-348; Enterokinase recognition site: bases 352-366; LacZ ORF: bases 373-3431; rrnB transcription termination region: bases 3518-3674; ampicillin ORF: bases 3953-4813; pMBI (pUC-derived)origin: bases 4958-5631; Ara C ORF : 7040-6162 Ara C ORF: 7040-6162

FIG. 2: Restriction enzyme map of pRA vector

FIG. 3: Map of pLMAB vector
LMAB promoter region: bases 2-335; DAC affinity tag: bases 336-461;
Enterokinase recognition site: bases 474-488; MCS: 488-525; rrnB transcription termination region: bases 607-764; Ampicillin^{r} ORF: bases 1043-1903; pMBI(pUC-derived) origin: bases 2721-2048; AraC ORF: bases 4130-3252

FIG. 4: Restriction enzyme map of pLMAB vector

FIG. 5: Kinetics of Induction of pBHL, pRAL and pLMAB-LacZ vector

FIG. 6: Fermentation profile of pLMAB-hGH grown in the 1 litre fermentor

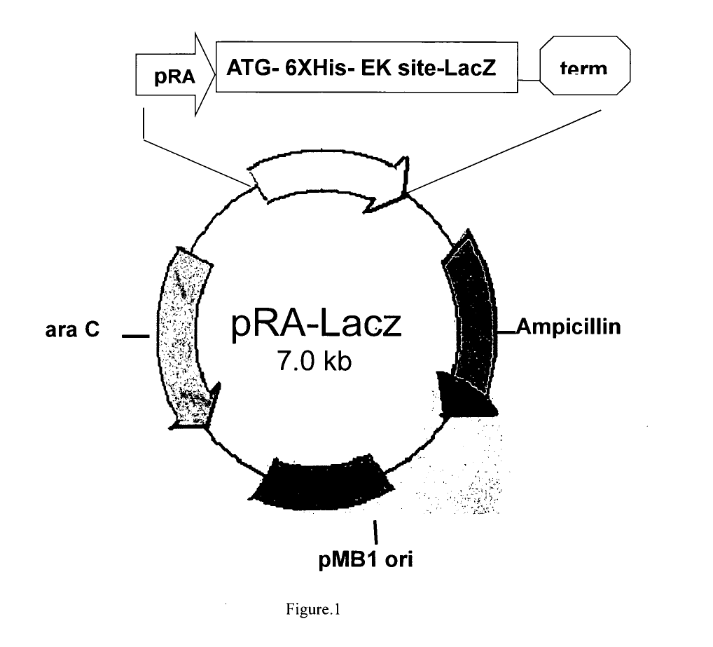

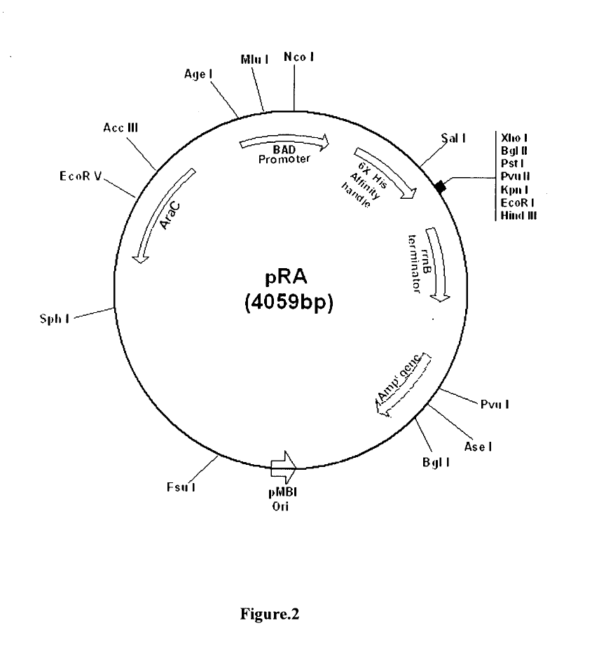

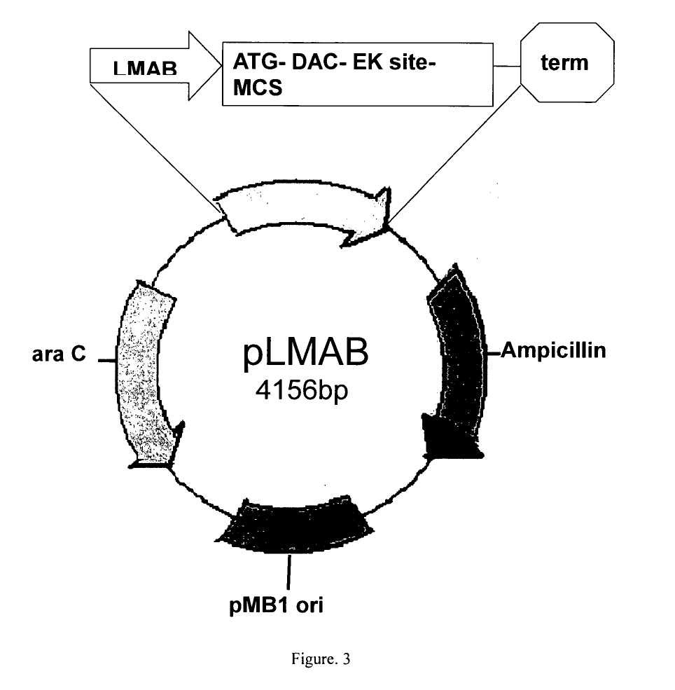

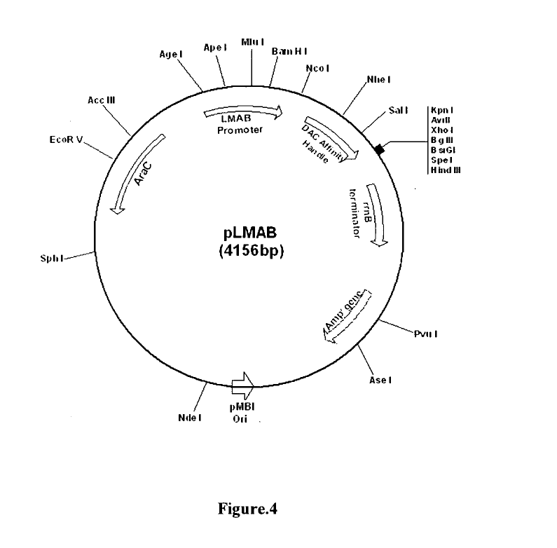

FIG. 7: SDS -PAGE profile showing expression of different proteins in the pLMAB vector grown in the 1 litre fermentor: Lane 1: Molecular weight marker;
Lane 2: Un-induced; Lane 3: hGH, Lane 4: EK, Lane 5: IL2; Lane 6: PDGF;
Lane 7: Rete; Lane 8: OmpC; Lane 9: Molecular weight marker; Lane 10: Molecular weight marker.

### DETAILED DESCRIPTION OF THE INVENTION

A variety of expression vectors are now available commercially. Most use moderate to high copy number plasmids. These can drive rapid protein expression, but usually require active selection pressure, for example by antibiotics. However, a case can be made for low copy number plasmids or for expression cassettes incorporated into the chromosome. (James R Swartz., Current Opinion in Biotechnology 2001, 12: 195-201).

In order to get optimal expression of a cloned gene, the type of promoter and ribosome-binding site (RBS) have to be taken into account in making a choice in favour of an expression vector. The first step in expressing eukaryotic proteins in bacteria is to choose an expression vector that has a strong constitutive or regulated prokaryotic promoter. Promoters have been classified as strong or weak, primarily on the basis of a comparison of the amounts of the gene product made. Some constitutive promoters of bacteriophage T3, T5 and T7 are considered to be strong promoters. The same is true for two highly controllable promoters, pL and pR, of the leftward and rightward operon, respectively, of bacteriophage A and for the regulatable promoter of the tryptophan (trp) operon of E. coli.

A variety of promoters are now used for protein expression. Yet, there is still a need for a promoter with little or no expression before induction and with reliable, adjustable expression. Possibly, the arabinose promoter system comes closest to fulfilling these objectives. The modified araB/pHO promoter control system with very tight regulation is the field of this invention. This promoter is induced with L-Arabinose and the protein coded by the heterologous gene linked to the promoter is not synthesized prior to addition of L-arabinose to the culture media. An essential feature of present invention is that upon induction with L-Arabinose, the protein is expressed efficiently in a short induction time of just 3 hours. The expression system under controlled conditions is sensitive to extracellular Pi concentration. The present invention provides a novel expression plasmid hereinafter called pLMAB, for expression of proteins under the control of an inducible promoter. The plasmid of the present invention may express and produce prokaryotic and eukaryotic proteins in E. coli . E. coli remains the system of first choice for expressing proteins, as it is cheap and easy to handle, however many mammalian proteins cannot be successfully expressed in E. coli. This leaves the researcher to either explore expression space using a range of alternative E. coli strains, different temperatures, solubility tags or choose an alternative expression host.

Once the gene or gene fragment is inserted in the expression vector, the protein product can be obtained in a suitable host strain that contains the genotypic features needed for promoter regulation and cell growth. The cloned vector is utilized to transform an appropriate host and the host is grown at 37° C. in a shaking incubator. After an appropriate period of time, the promoter is either constitutively expressed or induced with an inducer for certain time intervals and then the cell pellet is lysed and analysed either for enzymatic activity or by SDS-PAGE and Western Blotting.

E. coli plasmids are widely used in recombinant DNA-based biotechnologies as vectors for overproduction of heterologous proteins. Among the phenotypes conferred by these plasmids are resistance to antibiotics, production of antibiotics, degradation of complex organic compounds and production of colicins, restriction and modification enzymes.

Translational initiation is an important step in prokaryotic gene expression. The efficiency of translation is strongly affected by secondary structures around the site of initiation of translation. The promoter is a region on the gene to which RNA polymerase binds and initiates transcription to ultimately lead to formation of polypeptides. In E. coli, initiation of protein synthesis begins at a start codon, which in most cases is an ATG. This start codon is at the center of an RNA fragment, which is 30 to 40 bases in length called the ribosome binding site (RBS). In most RBS, the start codon is preceded by a 3-9 nucleotide long, purine-rich sequence at a distance of 5 to 12 bases called Shine-Dalgarno (SD) sequence which is complementary to the 3′ end of 16S rRNA and is thought to assist the RNA polymerase positioning at the proper place with respect to the start codon on the mRNA. The distance between the start codon and the Shine-Dalgarno sequence has been found to affect the efficiency of translation initiation process. Also, the sequences around these elements, including sequences downstream of the start codon appear to affect translational efficiency. Given a defined SD-ATG region, the major controlling factor in translational initiation is the nature and stability of the secondary structure in which it is involved, or by which it is surrounded. The intramolecular base pairings giving rise to secondary structure that can influence translational initiation may involve regions many nucleotides upstream of the SD region and/or downstream of the start codon. (Schuader, B. and McCarthy, J. E. G. (1989) Gene 78 : 59-72). It was shown that A or T residues following the SD region are favorable for the translation efficiency, whereas G residues and to a lesser extent, C residues were inhibitory to translation. (Hui, A., Hayflick, J., Dinkelspiel, K and de Boer, H. A. (1984) The EMBO Journal, 3 : 623-629). Ribosome accessibility to mRNA would be enhanced if the SD sequence and the ATG initiation codon were located in an AT-rich sequence free of local secondary structures. In some cases, the start codon ATG can be found in and out of the natural reading frame. Such pseudo-initiation sites can also affect the translational efficiency. A factor that may negatively affect translation of a cloned gene is the presence, in front of a genuine start ATG codon, of a second Shine-Dalgarno sequence and ATG codon. The ribosomal subunit binds initially at the 5′ end of mRNA and subsequently migrates until it reaches the first AUG triplet; if the first AUG codon occurs in an optimal sequence context, all subunits stop there and that AUG serves as the unique initiation codon. But if the first AUG triplet occurs in a suboptimal sequence context, only some subunits stop and initiate there, some bypass that site and initiate at another AUG that lies further downstream (Kozak, M (1984) Nature 308 : 241-246).

The efficiency of araBAD promoter was initially studied using a pRA-LacZ vector. To create pRA-LacZ expression vector (FIG. 1) after the initiation ATG (Met) of pRA-LacZ vector, nucleotide sequence of the 2nd amino acid has been changed from GGG to GGC (to increase the % of codon usage in bacteria from 12.3% to 45.6%) and 4th amino acid has been changed from TCT (Ser) to GCG (Ala). The 6th CAT of Poly His region has been changed from CAT to CAC so that the out of frame ATG has been removed. Sal I site has been created immediately after the polyHis region (There is no Nhe I site in the vector). Eleven amino acids i.e 33 bp from the region after start codon have been removed. The 2nd amino acid of EK Recognition site has been changed from GAT to GAC. This gets rid of the out of frame ATG. Part of the Express™ Epitope (GAT CTG TAC) has been deleted. The fourth codon of the EK Recognition site has been changed from GAT to GAC to get rid of the out of frame stop codon TAA. Another codon (details not disclosed) in the region after start codon has been changed from CGA to CGT to eliminate the out of frame start codon ATG and to increase the % of codon usage in bacteria from 5.6% to 36.9%. the pRA vector. The pRA vector (Seq ID 2) series of expression vectors differing in different enhancer sequences have been constructed by insertion of AT rich sequences between the transcription start site and the shine Dalgarno sequence. These vectors exhibit high level expression of difficult to express genes.

An essential feature of the present invention is construction of the pRA-LacZ vector with synthetic araBAD promoter from E. coli as shown in Seq ID 2 wherein the in and around surroundings of ribosome binding site have been modified as stated above to achieve smooth translation and high expression levels.

A large number of plasmid vectors have been constructed that contain powerful promoters that generate large amounts of mRNA complementary to cloned sequences of foreign DNA. These include the lactose promoter, beta lactamase (Chang et al, Nature, 1978, 275: 615; Itakura et al, Science, 1977, 198 : 1056; Goeddel et al, Nature, 1979, 281: 544), trp (tryptophan) promoter (Goeddel et al, Nucleic Acids Res., 1980, 8: 4057), deo promoter and tac promoter (a hybrid trp-lac promoter that is induced by adding IPTG, a relatively expensive compound). Other promoters include the bacteriophage λ promoters (pL and pR), which are regulated by shifts in temperature. This system is induced by incubating the cells at 42° C., which may lead to greater misfolding of the expressed protein.

Cosmid vectors, plasmids carrying a lambda phage cos site, were developed to facilitate cloning of large DNA fragments. Many cosmid vectors are between 5 to 10 kb in size and can therefore accept inserts of 30 to 45 kb. Cosmids can be transformed into cells like plasmids and once in the cells, replicate using their plasmid ori. Another type of plasmid cloning vector, called bacterial artificial chromosome (BAC) has been developed using the F factor replicator for propagation of very large pieces of DNA (100 to 500 kb). Plasmids have been developed that contain a filamentous phage origin of replication in addition to a plasmid ori. These “phagemid” vectors can be grown and propagated as plasmids. However, upon super-infection of a plasmid-containing cell with a wild-type helper phage, the phage ori becomes active and single-stranded DNA is produced and secreted.

Regulation of transcription initiation by proteins binding to DNA sequences at various distances from the transcription start site of the promoter seems to be an universal feature of both eukaryotes and prokaryotes. Proteins bound at an enhancer site can turn on genes at a distant site, whereas efficient repression of some prokaryotic genes like gal, ara and deo, operons of E. coli requires presence of more than one operator sites (Dandanell. G et al., 1987. Nature 325: 823-826). The L-Arabinose operon in E. coli i.e the ara BAD operon exhibits control of gene expression via two positive control components, the ara C protein- L-Arabinose complex and the cAMP receptor- cAMP protein complex. Both araC protein and cAMP receptor proteins are required for transcription initiation from the ara BAD promoter. The RNA polymerase and cAMP receptor protein binding site/recognition site of the ara BAD promoter is similar to those for galactose and lactose operons. The ara C protein activates the araBAD operon to high levels when it is present in cis rather than trans. However, araBAD promoter is known to have two classes of cis-acting constitutive mutations, one the aralc mutations which allow low level constitutive expression of araBAD in the absence of the positive regulatory araC gene product, and the ara Xc mutations which allow expression of araBAD in the absence of cAMP receptor protein (Horwitz A. H. et al., 1980.J. Bacteriology 142:659-667). The binding sites specific for araC protein, cAMP-binding protein and RNA polymerase have been determined by methylation protection and DNAse I protection methods. The promoter activity as measured by transcription initiation correlated by site occupancy of these sites. The araC protein either in its activator (P₂) or repressor (P₁) form was shown to be a repressor for araC at the RNA polymerase site of the araC promoter. araC and the araBAD promoter have been shown to share a common site of positive control by cAMP binding protein, located 90 bases from the araBAD and 60 bases from the araC transcription start points (Lee N. L. et al., 1981 Proc. Natl. Acad. Sci(USA) 78:752-756). The araC promoter is known to be derepressed to about 5 fold for 20 to 30 min post addition of arabinose to the fermentation medium, where in as a function of time, the araC promoter became progressively derepressable, whereas the araBAD promoter (pBAD) remained normally inducible (Hahn S. & Schleif R. 1983 J. Bacteriology 155: 593-600).

Deletion strains show that the pBAD promoter has two sets of domains for promoter induction by araC binding, one is at the +20 to −110 of the promoter and other between positions −265 and −294. Repressions were impaired in those cases where half-integral turns of DNA helix were introduced i.e. at −16, −8, +5, +24 and there was normal repression at 0, +11 and +31 base pairs (Dunn T. M et al., 1984 Proc Natl. Acad. Sci USA 81: 5017-5020). Deletions from the PC side of the CRP site located between −80 and −120 w.r.t the pBAD promoter transcription start site, reduced the activity of the promoter (Dunn T. M & Schleif R. 1984. J. Mol. Biol. 180: 201-204). Further, deletion mutation studies showed that the catabolite gene activator protein (CAP) has no role in relieving of repression (Lichenstein H. S. et al., 1987 J. Bacteriology 169: 811-822). Binding of CAP induced a bend in the ara DNA as it does for the lac DNA (Lichenstein H. S et al., 1987 J. Bacteriology 169: 811-822 and Huo L et al., 1988. Proc Natl. Acad. Sci USA 85: 5444-8). This DNA loop formation has been proposed as a common regulatory mechanism explaining both repression and catabolite gene activation. Two different DNA loops are formed in the ara regulatory regions when the ara C protein binds two different DNA regions as well as to each other. Mutational studies show that N-terminal half of ara C is essential for the formation of the DNA loops for autoregulation of araC and repression of araBAD (Menon. K. P & Lee N. L. 1990 PNAS, USA 87: 370812). Of the arabinose inducible promoters tested, araFGH promoter is more catabolite sensitive (Hendrickson W. et al., 1990 J. Mol. Biol. 215: 497-510) than the other ara promoters (ara E, ara J, ara BAD). The mechanisms for araBAD and other arabinose inducible promoters have been investigated in high level production of proteins (Guzman et al., 1995. J. Bacteriol. 177:4121-4130; Zhang. X et al., 1996. J. Mol. Biol. 258: 14-24)

The current investigation aims at construction of synthetic hybrid arabinose inducible promoters in the context of expression of recombinant peptides and proteins in the E. coli host.

Three arabinose-inducible operons of E. coli—araBAD, araE and araFGH have been identified and studied (Hendrickson W. et al, J. Mol. Biol. (1990) 215: 497-510). The araBAD operon codes for genes that are responsible for the catabolism of L-Arabinose. Genes in the araFGH and araE operon code for the arabinose binding protein and additional proteins involved in the high affinity transport system. These operons are co-ordinately controlled by the inducer L-arabinose and the araC regulatory gene product. Adjacent to the araBAD operon is a complex promoter region and the regulatory gene araC. The araBAD and araC genes are transcribed in opposite directions. Within and around the promoters for araBAD (P_{BAD}) and araC (P_{c}) lie binding sites for the AraC protein, the cyclic AMP receptor protein (CRP) and RNA polymerase. Alone or in combination; proteins bound to these regions both in the presence or absence of the inducer L-arabinose, tightly regulate expression from both promoters. Use of araB promoter in a vector for producing polypeptides is already known. Very low levels of transcription from pBAD occurs in the absence of arabinose. In the presence of arabinose, AraC protein binds at the aral site immediately adjacent to the RNA polymerase binding site of the araB promoter and stimulates transcription of the araBAD operon. In the absence of arabinose, the AraC protein represses mRNA synthesis from the promoter by a mechanism involving the formation of a DNA loop. Without arabinose, most copies of the ara regulatory region contain a DNA loop between the araO₂ and aral sites mediated by AraC protein bound to both of these sites. This loop constrains AraC protein bound at aral from entering the inducing state and holds the uninduced levels low. Upon the addition of arabinose, the araO2-aral loop opens, and arabinose bound to AraC protein on the aral site drives AraC into the inducing conformation, thereby inducing P_{BAD}.

The araC protein is a regulatory protein that exerts positive and negative effects on the various operons that make up the arabinose regulon in E. coli. In the presence of arabinose and in conjunction with the cAMP receptor protein (CAP), it stimulates transcription of the araBAD, araE and araFGH operons. The AraC protein also autoregulates its own gene and represses transcription from the araBAD operon (Francklyn, C. S. and Lee, N. (1988) J. Biol. Chem. 263 (9): 4400-4407). Transcriptional activation of the araBAD promoter by AraC requires the binding of the protein to the initiator site located within the promoter. Regulation of this operon is also subject to catabolite repression, so even in the presence of arabinose, significantly less induction occurs when intracellular cAMP levels are low, such as when the cells are grown in the presence of glucose. Presence of glucose reduces the uninduced levels even further.

Another operon that responds to extracellular culture component and tightly regulated is the pho operon regulatable by phosphate concentration (Pi). The pho regulon includes more than 31 genes arranged in eight separate operons, all of which are co-regulated by extracellular Pi. Inorganic phosphate (Pi) is the preferred phosphorous source for E. coli. When Pi is not available, an adaptive response is activated which includes about 50 proteins involved in scavenging other forms of phosphates such as organic phosphates or in utilizing other P sources. Many of the genes encoding proteins that are part of the adaptive response are members of a single regulatory network (the PHO regulon) which is defined by the involvement of a 2-component regulatory system, PhoR and PhoB. Several of these responders were found to contain a sequence in their promoter region similar to the sequence called phoB box (ctttxxxcat (atyyyat) ctttdddcac). The phoA gene of E. coli is a structural gene for alkaline phosphatase which is induced upon derepressed levels of Pi (Berg P. E, 1981. J. Bacteriol. 146(2):660-667). The phoR gene product functions as a negative regulator in presence of increased Pi and as a positive regulator with limited phosphate for the phosphate-starvation-inducible pho regulon in E. coli. The phoB and phoR constitute a single operon whose promoter is located proximal to phoB and maximal level of the operon is inducible as a result of increased phoR protein and of functional change of the protein as a positive regulator induced by phophate limitation (Makin K. et al., 1985 J. Mol. Biol. 184(2:231-240). The phoR is transcribed from its own promoter in presence of excess phosphate and during phosphate limitation, phoR is dependant on the upstream phoB promoter. It is shown that phoB protein which is the transcriptional activator for the pho regulon, protected the regulatory region with the pho-box and activated transcription from the downstream promoter in vitro as found by DNase I footprinting experiments (Kasahara M. et al., 1991. J. Bacteriol. 173(2)549-558). Thus phoB is a transcription activator while the phoR is a phosphate sensing protein (Wanner B. L & Chang B. D. 1987. J. Bacteriol. 169(12):5569-5574). Another protein that is turned on under phosphate starvation is the phoE gene product, an outer membrane pore protein, whose expression is induced under phosphate limitation. The promoter of this gene contains a 17 bp pho-box which is also found in other phosphate-controlled promoters. The sequences upstream of the pho-box (−106 to −121) are required for the efficient expression of phoE (Tommassen J. et al., 1987. J. Mol. Biol. 198(4):633-41). The concensus pho-Box is defined as two direct repeats spaced by four bases which are part of the −55 to −35 region of the promoters and end 10 bases upstream of the beginning of the −10 region of the promoter (VanBogelen, R. A., Olson, E. R., Wanner, B. L. and Neidhardt, F. C (1996)J. Bacteriology 178 (15:4344-4366).

Our invention is focused on the effect of insertion of synthetic consensus pho-box sequence variants as well as its variants (8-35 bp sequences) in modulating transcription from the promoters of the ara-operon. A series of pho box sequence variants as shown in Seq ID 15 top Seq ID 22 form an essential feature of invention. Surprisingly due to introduction of above pho box sequence variants in synthetic araBAD promoter in −55 to −10 region and RBS, the induced expression increased 100 fold over the uninduced one. Most surprisingly within short period of 3 hours after induction nearly 62% of final expression was obtained which remained consistent till the end of the fermentation run. Also the stability of the expression vehicle constuct is another feature of our invention. Deposit of expression vehicle harbored in E. coli Top 10 host strains for patent purposes under Budapest treaty is made with American type culture collection, USA on Aug. 21, 2008 and has accession number as PTA-9371.

Such synthetic promoters were shown to be regulated in a very tight way by the preferred disclosures of this invention. During Pi limitation, phoR turns on genes of the PHO regulon by phosphorylating phoB which in turn activates transcription by binding to promoters that share the 18-base concensus PHO box. When Pi is in excess, PhoR , Pst and PhoU together turn off the PHO regulon by dephosphorylatory phoB with Cre C and acetyl phosphate being directly involved in phosphorylating pho B (Wanner B. L 1993 J. Cell Biochem 51(1):47-54). Another pho regulon gene is the phoH gene. The promoter for phoH has two sites P1 & P2. Whereas the P1 promoter site requires the pho B function and was induced by phosphate limitation, the transcription from the P2 promoter was constitutive and independent of phoB (Kim S. K et al., 1993. J. Bacteria 175(5):1316-1324). The members of phosphate transport system (Pst) consists of four genes- Pst A, PstB, Pst C and Pst S which are all known to negatively affect the PHO regulon (Haldimann A. el al., 1998. J. Bacteriol. 180(5):1277-86). The transcription of the PHO regulon genes is initiated by the RNA polymerase complexing with sigma D (Taschner N. P et al., 2006. Arch. Microbial. 185(3): 234-237).

A number of factors play a role in expressing heterologous proteins in E. coli such as choice of host, plasmid copy number, strength of the promoter, effectiveness and spacing of transcription terminator and stability of the mRNA (secondary structure especially at the 5′ end of the message often plays a critical role). The positioning of the translation signal with respect to RBS affects the level of ribosome binding & clearance and hence expression. Also, secondary structure at the 5′ end of the message can affect the accessibility of the RBS. Optimal codon usage, temperature of growth, and growth conditions like oxygen levels, Carbon source, growth rate & fermentor configuration affects expression. (S. Jana & J. K. Deb, Appl. Microbiol. Biotech. (2005) 67 : 289-298).

Many expression systems in research and industry use plasmids as vectors for the production of recombinant proteins. Plasmids have an essential impact on productivity and factors that affect production are plasmid copy number, structural plasmid stability and segregational plasmid stability. Plasmid copy number reflects the average number of copies of a plasmid inside a host cell and determines the gene dosage accessible for expression. Many plasmids generally lead to a high productivity. To analyze an expression system the quantification of plasmid copy number is very helpful. Researches usually choose high copy number plasmids as their vectors since one can get a large number of plasmids from relatively fewer cells in less time. Nevertheless, to ensure a high production of recombinant proteins, it is necessary to maintain an optimal plasmid copy number in bacterial cells. This level must be sufficient for the desired gene dosage effect, yet not so high that it induces metabolic

burden and loss of cell resources. Construction of synthetic hybrid promoter with inbuilt pho box element synthetic sequence to enhance protein expression and vector stability and host compatibility is an essence of the present invention.

The most common block to efficient expression of foreign genes is poor translation initiation. The E. coli ribosome often does not recognize the chimeric junction between a prokaryotic ribosome binding site and a foreign coding region. This can be overcome by having part of an E. coli gene upstream of the foreign gene, which is usually made at high levels because transcription and translation initiation are directed by normal E. coli sequences. Also, foreign proteins are often rapidly degraded by host proteases and this may be avoided by a gene fusion strategy. Using affinity handles as fusion partners, efficient purification schemes may be used which allows rapid recovery of foreign gene products. In addition, the foreign proteins can be localized to different compartments of the host cell through specific peptides fused to the protein.

A lot of efforts have been put in optimizing expression systems in the context of the production process to improve overall yields and efficiencies. A number of alternate expression systems is also being developed and evaluated, not all of which will be useful for the production of therapeutic protein production. The use of E. coli has many advantages which have ensured that it remains a valuable organism for the high level production of recombinant proteins. A variety of procaryotic expression vectors are now available commercially.

The minimal elements that an expression plasmid vector should have are a well-characterised origin of replication and a selection marker for plasmid propagation and maintenance; a strong promoter (usually regulatable); a ribosome binding site and a translation initiation ATG codon.

The present invention describes the expression of recombinant proteins from LMAB promoter, a novel, synthetic hybrid promoter of ara & pho elements juxtaposed functionally and modifying the region around the ribosome binding site (RBS) which is critical for high yield expression (SDS-PAGE data not included) of heterologous proteins.

One of the embodiment of the present invention is an expression vehicle comprising an isolated nucleic acid as shown in Seq. ID No. 1 comprising of a synthetic hybrid promoter wherein the hybrid promoter comprises of an inducible arabinose promoter derived from E. coli and a synthetic stretch of 8-35 nucleotides from pHO regulon introduced in the region between −10 and ribosome binding site from the transcription initiation site.

Another embodiment of the present invention is that in the isolated nucleic acid, the inducible arabinose promoter sequence consists of a stretch of 17-32 nucleotides introduced in the region between −55 to −10 and ribosome binding site from the transcription initiation site selected from the group consisting of Seq ID No. 15, Seq ID No. 6, Seq ID No. 17, Seq ID No. 8, Seq ID No. 19, Seq ID No. 20, Seq ID No. 21 and Seq ID No. 22.
Still another embodiment of the invention is an expression vector comprising a synthetic hybrid promoter as shown in Seq ID No. 1.
Still another embodiment of the present invention is an expression vector , wherein the inducible arabinose promoter sequence consists of a stretch of 17 nucleotides as shown in Seq. ID No. 15, introduced in the region between −10 and ribosome binding site from the transcription initiation site operably linked to heterologous protein characterized in that the expression is induced after 3 hours of induction. Still another embodiment of the invention is an expression vector, wherein the inducible arabinose promoter sequence consists of a stretch of 17 nucleotides as shown in Seq. ID No. 15, introduced in the region between −10 and ribosome binding site from the transcription initiation site operably linked to heterologous protein wherein at least 100 fold protein expression is obtained as compared to the yield obtained under uninduced conditions.
Also another embodiment of the present invention is a prokaryotic host cell harboring the expression vehicle comprising an isolated nucleic acid as shown in Seq ID No. 1 for the increased expression of heterologous genes coding for polypeptides.
Still another embodiment of the invention is a prokaryotic host cell harboring the expression vehicle comprising an isolated nucleic acid as shown in Seq ID No. 1 with an ATCC accession no. PTA-9371 for the increased expression of heterologous genes coding for polypeptides.
Translation initiation region is defined as that stretch of mRNA controlling the efficiency of initiation.
Transcription initiation site is defined as the first nucleotide of a transcribed DNA sequence where RNA polymerase (DNA-DIRECTED RNA POLYMERASE) begins synthesizing the RNA transcript.

pRA (Seq ID 2) and pLMAB (Seq ID 1) are expression vehicles constructed with an inhouse nomenclature and FIG. 1 and FIG. 3 represents respective maps of the same. Both the expression vehicle consists of synthetic arabinose promoter with region around the ribosome binding site modified. pLMAB has an additional feature with the introduction of pHO box element sequence in −55 to −10 and ribosome binding site from the transcription initiation site.

### GLOSSARY

lac: Lactose
E. coli: Escherichia coli
trp: tryptophan
pL and pR: Promoter left and promoter right
pho: phosphate
ara: arabinose
CT-B: Cholera toxin subunit B
RBS: Ribosome-binding site
Pi: inorganic phosphates
SD: Shine Dalgarno sequence
SDS-PAGE: Sodium Dodecyl Sulphate polyacrylamide gel electrophoresis
IPTG: Isopropyl β-D-1-thiogalactopyranoside
ori: origin of replication
BAC: Bacterial Artificial Chromosome
cAMP: Cyclic adenosine monophosphate
CAP: Catabolite Activator Protein
CRP: Cyclic AMP receptor protein
Pst: phosphate transport system
ONPG: O-nitrophenyl β-D-Galacto-Pyranoside
β-Gal: β-Galactosidase
YE: Yeast extract
The following examples are given for illustrating the present invention and are not limiting the scope of the present invention.

### EXAMPLES

### Example 1

### PCR Amplification Process

The araB promoter, araC gene and the araC regulatory region were amplified from E. coli (TOP-10/BL-21/HB101/JM109) genomic DNA. For all PCRs, amplification was done using specially designed primers, genomic DNA template and Pfu polymerase enzyme (1-2.5 units/ul, MBI) under different cycling conditions (eg. 25-35cycles of denaturation at 95° C. for 1-2min, annealing at 45° C. -70° C. for 1-2min and extension at 72° C. for 1-2min). For analysis of the PCR products, 5-10 μl of sample was mixed with 5-10 μl of 1× Loading Dye and run on 0.8%-1.5% Agarose Gel as required. Digestion with a restriction enzyme (such as Ssp I, Age I, Ase I etc.) was done in respective 1× buffer at 30° C.-37° C. for 2 hrs-overnight. The digested DNA was run on an agarose gel (0.8-1.5%) containing ethidium bromide and the desired fragments were cut out from the gel. The agarose was dissolved in sodium iodide solution at 50° C. -60° C. and the DNA was purified using Qiaquick PCR purification kit (Qiagen). DNA sample to be purified was mixed with 5 volumes of the Buffer PB provided in the kit and applied to a Qiaquick column. This was spun for 30-60 secs at 14K and the flow through was discarded. The membrane was washed with the wash buffer provided and the DNA was eluted with nuclease free distilled water. Ligation was done using T4 Ligase enzyme in ligation buffer containing 40 mM-50 mM Tris-HCl, 10 mM MgCl₂, 1 mM-10 mM DTT and 0.5 mM-1 mM ATP (pH 7.6-7.8) at R. T/37° C. for 20 mins- 2 hrs followed by an overnight incubation at 4° C.-12° C.

### Example 2

Construction of pLMAB Vector

Using E. coli JM109 DNA as the template, 2 PCRs were carried out with specially designed primers—PCR I with forward primer Seq ID 3 and reverse primer Seq ID 4 and PCR II with forward primer Seq ID 5 and reverse primer Seq ID 6. Pfu polymerase enzyme(1-2.5 units/μl, MBI) was used for the amplification under following cycling conditions (eg. 25-35cycles of denaturation at 95° C. for 1-2 min, annealing at 45° C. -70° C. for 1-2 min and extension at 72° C. for 1-2 min).

The PCR products were purified and digested with a restriction enzyme ‘A’ (Apo I). The digested fragments were purified and ligated. The 1550 bp ligated fragment 1 was purified and used as a template for amplification with primers Seq ID 7 & Seq ID 8.

The 1512 bp PCR product was purified, digested with restriction enzymes ‘B’ (Sph I) & ‘C’ (Nco I) and the digested 1482 bp fragment 2 was purified. pDAC-LacZ vector (has the novel affinity handle and β-Galactosidase gene as the reporter gene) was also digested with restriction enzymes ‘B’ (Sph I) & ‘C’ (Nco I) and the digested 5852 bp fragment 3 was purified. Fragment 2 and Fragment 3 were ligated to create an intermediate vector-pLMAB-I(has bacterial AraB promoter, AraC gene, AraC regulatory region and our inhouse affinity tag, DAC). pLMAB-I vector was digested with Ssp I, run on a 1% agarose gel and the 2055 bp Fragment 4 was purified from the gel. Using Fragment 4 as the template, 2 PCRs were carried out with specially designed primers—PCR I with forward primer Seq ID 9 and reverse primer Seq ID 10 and PCR II with forward primer Seq ID 11 and reverse primer Seq ID 12. Pfu polymerase enzyme (1-2.5 units/ul, MBI) was used for the amplification under following cycling conditions (eg. 25-35 cycles of denaturation at 95° C. for 1-2 min, annealing at 45° C. -55° C. for 1-2 min and extension at 72° C. for 1-2 min).

The 323 bp PCR I product was purified and an A was added to the 3′ end with Taq polymerase in the presence of dATP & the A-tailed Fragment 5 was purified. Similarly, the 635 bp PCR II product was purified and a T was added to the 3′ end with Taq polymerase in the presence of dTTP & the T-tailed Fragment 6 was purified. Fragment 5 and Fragment 6 were ligated and the 958 bp ligated Fragment 7 was purified. This was digested with restriction enzymes ‘D’ (Age I) & ‘E’ (NcoI) and the digested 237 by Fragment 8 was purified. pLMAB-I vector was also digested with restriction enzymes ‘D’(Age I) & ‘E’ (NcoI) and the digested 7093 by Fragment 9 was purified. Fragment 8 and Fragment 9 were ligated to create pLMAB-LacZ consisting of synthetic arabinose hybrid promoter, linked to the affinity polypeptide which in turn is linked to EK site which is further linked to β-Galactosidase gene controlled by LMAB promoter. The map of pLMAB vector is shown in FIG. 3. Seq ID 1 represents pLMAB vector sequence which has the additional pHO element sequence as shown in Seq ID 15 in the araB promoter as compared to the pRA vector sequence as shown in Seq ID 2. Seq ID 16 to Seq ID 22 show pHO box sequence variants which mimic pHO box element sequence as shown in Seq ID 15.

### Example 3

Preparation of pLMAB-hGH

The recombinant vector pRA-hGH (containing the ORF of human growth hormone) was digested with Sal I & Pvu I in buffer containing 33 mM Tris-acetate, pH 7.9, 10 mM Mg-acetate, 66 mM K-acetate and 0.1 mg/ml BSA at 37° C. overnight and the digested 1549 bp Fragment 10 was purified. The novel vector pLMAB was also digested with Sal I & Pvu I and the 3157 bp Fragment 11 was purified. Ligation of Fragment 10 with Fragment 11 was carried out using 2 units of T4 DNA Ligase in presence of buffer containing 40 mM Tris Hcl, pH 7.8, 10 mM MgCl2, 10 mM DTT and 0.5 mM ATP at 37° C. for 2 hrs and overnight at 4° C. to produce pLMAB-hGH vector (4706 bp) which has the affinity handle linked to the EK site which is further linked to hGH gene controlled by LMAB promoter.

### Example 4

Preparation of pLMAB-EK

The recombinant vector pRA-EK which has the ORF of bovine Enterokinase was digested with Sal I & Pvu I in buffer containing 33 mM Tris-acetate, pH 7.9, 10 mM Mg-acetate, 66 mM K-acetate and 0.1 mg/ml BSA at 37° C. overnight and the digested 1702 bp Fragment 12 was purified. The novel vector pLMAB was also digested with Sal I & Pvu I and the 3157 bp Fragment 11 was purified. Ligation of fragment 11 with Fragment 12 was carried out using 2 units of T4 DNA Ligase in presence of buffer containing 40 mM Tris Hcl, pH 7.8, 10 mM MgCl2, 10 mM DTT and 0.5 mM ATP at 37° C. for 2 hrs and overnight at 4° C. to produce pLMAB-EK vector (4859 bp) which has the affinity handle linked to the EK site which is further linked to EK gene controlled by LMAB promoter.

### Example 5

Preparation of pLMAB-Rete

The recombinant vector pRA-Rete having the ORF of human reteplase was digested with Sal I & Hind III in buffer containing 33 mM Tris-acetate, pH 7.9, 10 mM Mg-acetate, 66 mM K-acetate and 0.1 mg/ml BSA at 37° C. overnight and the digested 1154 bp Fragment 13 was purified. The novel vector pLMAB was also digested with Sal I & Hind III and the 4098 bp Fragment 14 was purified. Ligation of fragment 13 with Fragment 14 was carried out using 2 units of T4 DNA Ligase in presence of buffer containing 40 mM Tris Hcl, pH 7.8, 10 mM MgCl2, 10 mM DTT and 0.5 mM ATP at 37° C. for 2 hrs and overnight at 4° C. to produce pLMAB-Rete vector (5252 bp) which has the affinity handle linked to the EK site which is further linked to Reteplase gene controlled by LMAB promoter.

### Example 6

Preparation of pLMAB-IL2

The recombinant vector pRA-IL2 containing the ORF of human interleukin was digested with Sal I & Hind III in buffer containing 33 mM Tris-acetate, pH 7.9, 10 mM Mg-acetate, 66 mM K-acetate and 0.1 mg/ml BSA at 37° C. overnight and the digested 418 bp Fragment 15 was purified. The novel vector pLMAB was also digested with Sal 1 & Hind III and the 4098 bp Fragment 14 was purified. Ligation of Fragment 14 with Fragment 15 was carried out using 2 units of T4 DNA Ligase in presence of buffer containing 40 mM Tris Hcl, pH 7.8, 10 mM MgCl2, 10 mM DTT and 0.5 mM ATP at 37° C. for 2 hrs and overnight at 4° C. to produce pLMAB-IL2 vector (4516 bp) which has the affinity handle linked to the acid cleavage site which is further linked to Interleukin-2 gene controlled by LMAB promoter.

### Example 7

Preparation of pLMAB-GCSF

The recombinant vector pRA-GCSF containing the ORF of human GCSF was digested with Sal I & Hind III in buffer containing 33 mM Tris-acetate, pH 7.9, 10 mM Mg-acetate, 66 mM K-acetate and 0.1 mg/ml BSA at 37° C. overnight and the digested 554 by Fragment 16 was purified. The novel vector pLMAB was also digested with Sal I & Hind III and the 4098 bp Fragment 14 was purified. Ligation of Fragment 14 with Fragment 16 was carried out using 2 units of T4 DNA Ligase in presence of buffer containing 40 mM Tris Hcl, pH 7.8, 10 mM MgCl2, 10 mM DTT and 0.5 mM ATP at 37° C. for 2hrs and overnight at 4° C. to produce pLMAB-GCSF vector (4652bp) which has the affinity handle linked to the acid cleavage site which is further linked to GCSF gene controlled by LMAB promoter.

### Example 8

Preparation of pLMAB-OmpC

Using a vector pPROEXHTa (has the gene coding for the Omp C outer membrane protein of S. typhi under the control of T7 promoter) as the template, carried out a PCR with primers SEQ ID 13 & SEQ ID 14. The 1194 bp PCR product was purified, digested with Sal I & Hind III and the digested 1095 bp Fragment 17 was purified. Digested pLMAB vector with Sal I and Hind III and purified the 4098 bp Fragment 14. Ligated fragment 1 with Fragment 2 to create pLMAB-OmpC vector (5190 bp) which has the affinity polypeptide linked to EK site which is further linked to OmpC gene controlled by LMAB promoter.

### Example 9

### Transformation of Cells

Once the desired vector was constructed, competent bacterial hosts (BL21, Top 10, LMG194, HB101, JM109, G1724 cells) were transformed with the ligation mix. Required amount of competent cells of E. coli strains were thawed on ice. Ligation mix was transferred into the tube containing the competent cells, mixed gently and incubated on ice for 30 min. Cells were subjected to heat shock at 42° C. for 2 mins and incubated on ice for 5 mins. One ml of appropriate medium without antibiotic was added and cells were grown at 37° C. for 1 hr with shaking. Cells were pelleted at 3000 rpm/5 mins, resuspended in 100 μl appropriate medium without antibiotic and spread on an agar plate containing appropriate antibiotic. Colonies obtained on the agar plate were innoculated in 3 m1 liquid media and allowed to grow at 37° C. overnight with shaking. Plasmid DNA (Miniprep) was extracted by alkaline lysis method from 1.5 ml o/n cultures

### Example 10

### Plasmid Isolation and Analysis

Plasmid DNA was isolated by alkaline lysis method from 1.5 ml overnight grown cultures by standard methods in prior art. The miniprep DNAs were subjected to restriction enzyme digestions to confirm the vector construction. The DNA of interest was cleaved with a variety of restriction endonucleases, either individually or in combination and the resulting products were separated by agarose gel electrophoresis. By determining the sizes of DNA fragments produced by the action of the endonucleases, the restriction map was deduced progressively from simple situations where enzymes cleave the DNA once or twice to more complex situations where cleavage occurs more frequently.

Positive clones were sequenced using the automated DNA sequencer (ABI Prism 310 Genetic Analyzer). The plasmid was purified either through columns or by PEG precipitation. To the purified DNA, 4-8 μl of the terminator ready reaction mix was added. This mix is composed of premixed dNTPs, dye terminator, Taq DNA polymerase, MgCl2 and buffer. On addition of 1 μl of primer (5 pmoles/ul), samples underwent cycle sequencing in a thermal sequencer (25 cycles of 94° C. for 10 secs, 50° C. for 5 secs and 60° C. for 4 mins). The resulting products were precipitated with 2.7 M sodium acetate (pH 4.6) and ethanol. The resultant pellet was washed twice with 70% ethanol, air dried and dissolved in formamide. Samples were analyzed in the automated sequencer.

### Example 11

Expression Analysis of pLMAB-LacZ, pLMAB-hGH, pLMAB-EK, pLMAB-Rete, pLMAB-IL2, pLMAB-GCSF and pLMAB-OmpC

The bacterial cells which were transformed with the recombinant expression vector (pLMAB-LacZ, pLMAB-hGH, pLMAB-EK, pLMAB-Rete, pLMAB-IL2, pLMAB-GCSF and pLMAB-OmpC) were grown in liquid media in presence of appropriate antibiotic. The cells were grown either as a log (till the O.D₆₀₀ₙₘ reaches ˜0.5) or a stationary phase culture (16 hrs growth) at 37° C. in an incubator shaker or 1 lit fermentor. Then an appropriate amount (0-7% w/v) of the inducer was added to the culture and incubated further for required time (0-72 hrs) at 37° C. with shaking. The bacterial cells were harvested by pelleting at 4000 rpm for 10 mins at 4° C. The bacterial pellet was washed 3× with ice-cold 1× PBS and resuspended in 200 ul of 1× PBS. Bacterial cell extracts were prepared either by subjecting the cells to four cycles of rapid freezing in liquid nitrogen, followed by thawing at 37° C. and then vigorous vortexing for 5 mins or the cell pellets were sonicated in lysis buffer. Spun at 14000 rpm for 10 mins at 4° C. Transferred the supernatant to a fresh 1.5 ml eppendorf tube. Total protein of the cell extract was estimated by Bradford Method. Estimation of β-Galactosidase protein (for pLMAB-LacZ vector) was done colorimetrically using 0-nitrophenyl β-D-Galacto-Pyranoside (ONPG, Sigma)as the substrate and determining the O.D at 405 nm.

For the other vectors, the required amount of sample was mixed with sample buffer and heated at 90° C. for 5 mins. The samples were pulse spun and loaded on a SDS-PAGE gel (10-20%) immediately. After the electrophoretic run, the gel was stained with either Coomassie blue or silver or transferred to a blot for Western blot analysis. Table 1 depicts percent expression of pLMAB-LacZ in different strains of E. coli using 0.2% w/v of L-arabinose as inducer and Table 2 gives a comparison of percent expression in induced as well as uninduced condition in pLMAB and pBAD (commercial vector from Invitrogen) expression vectors.

TABLE 1
Percent expression of pLMAB-LacZ in different bacterial hosts

| | B-Gal conc./μg protein of cell |
|---|---|
| Bacterial strains | extract |
| Top 10 | 17.127 |
| JM109 | 13.698 |
| HB101 | 7.410 |
| G1724 | 9.646 |
| LMG194 | 10.433 |

TABLE 2
Expression levels of β-Galactosidase protein in the
pLMAB and pBAD vectors

| | B-Gal conc./μg Protein |
|---|---|
| Sample | of cell extract* |
| pBAD-His-LacZ (pBHL) Uninduced | 0.007 |
| pBHL-0.2% L-Ara Induced | 11.762 |
| pLMAB-LacZ-Uninduced | 0.230 |
| pLMAB-LacZ-0.2% L-Ara Induced | 20.412 |

*Average value from 4 experiments

Expression of LacZ gene in the novel procaryotic vector pLMAB as determined by Galactosidase activity was found to be 1.73 fold higher than that in pBAD/His vector.

### Example 12

E. coli strain TOP10 transformed to express the respective recombinant protein, (e.g., rhGH, rhIL2, etc.) was maintained in glycerol stocks. An aliquot of the culture was removed from the stock and streaked on 2.5% yeast extract medium (containing 50 mcg/ml ampicillin) plate to separate single colonies after growth of 24 hours at 37° C. A single colony from the plate was inoculated into 10 ml of 2.5% YE liquid medium contained in a falcon tube. After growth for 16 hours at 37° C. on a rotary shaker (200-220 rpm), 5 ml of the culture from the tube was inoculated into a 500 ml conical flask containing 100 ml of the basal medium. After growth for 8 hours at 37° C. on a rotary shaker (200-220 rpm), 100 ml of the culture from the flask was used to inoculate a jar fermenter (2 litres, B Braun) containing 900 ml of the basal medium.

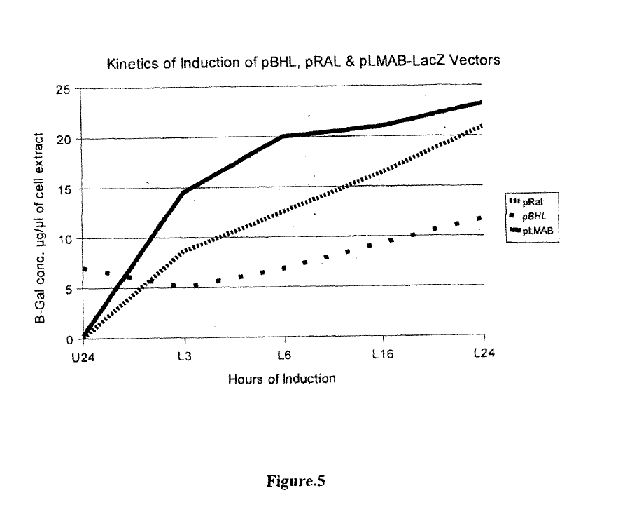

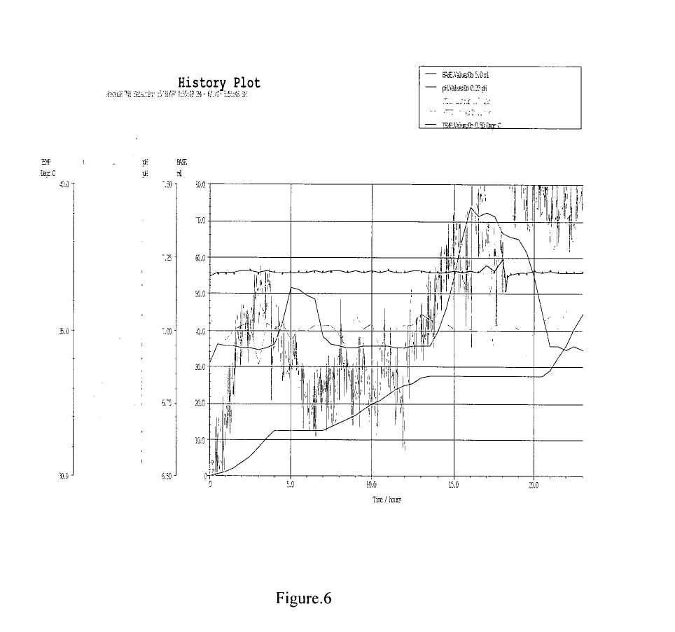

Top10 cells transformed with the 3 vectors: pBAD-His/LacZ (pBHL), pRA-LacZ(pRAL) and pLMAB-LacZ were induced with the inducer L-arabinose and cells assayed for β-galactosidase concentration/μg protein of cell extract at different time interval i.e 3 hrs (L3), 6 hrs(L6), 16 hrs(L16) and 24 hrs(L24) after addition of 0.2% w/v of L-arabinose as inducer with respect to the control (U24) which was uninduced for 24 hrs. pLMAB-LacZ was found to have a faster rate of induction which remained high throughout the 24 hrs induction. At 3 hrs(L3), expression of β-galactosidase was ≈3.0 fold higher as compared to pBHL(FIG. 5). Table. 3 shows Induction kinetics at different time intervals with 3 different vectors given below,

TABLE 3

| | Time of induction | | | | |
|---|---|---|---|---|---|
| | L3 | L6 | L16 | L24 | U24 |
| | B-galactosidase | | | | |
| Clone | concentration/μg Protein of cell extract | | | | |
| pRAL | 8.65 | 12.50 | 16.36 | 20.87 | 0.03 |
| pBHL | 5.06 | 6.84 | 9.30 | 11.76 | 0.01 |
| pLMAB-LacZ #12* | 14.50 | 20.00 | 20.97 | 23.19 | 0.21 |

*#12 signifies clone no. 12 from pLMAB-LacZ series

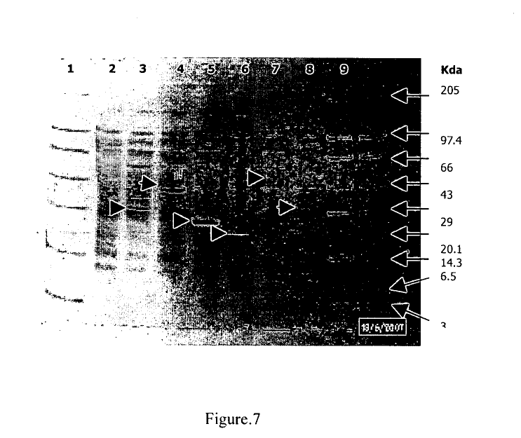

Fed-batch fermentations were carried out for all the clones in order to express large quantities of the recombinant protein at low to medium cell densities. Fermentation was carried out at a temperature of 37° C. and pH of the fermentation broth was maintained at pH 7 using 12.5% of ammonia solution. The stirrer was set at the maximum revolutions per minute (rpm) possible. When OD₆₀₀ of approximately 1 was reached or 2 hours after fermentation was started, the feed medium comprising glucose (carbon source), yeast extract (nitrogen source) and trace elements solution (2.5% v/v of the feed medium) was fed into the fermenter at a predefined feed rate. The glucose to yeast extract ratio was different for different clones as shown in Table 4. After 8 hours from the start of fermentation or when a cell concentration of OD₆₀₀ 15 was obtained, the fermenter was fed with an inducer solution containing 10 g of the inducer (arabinose) between OD₆₀₀ of 15 to 40. Excessive foaming was controlled with the addition of antifoam solution (Dow Corning 1510, Antifoam).The fermentation was carried out for 20-22 hours and during that time samples were taken for measurement of optical density and accumulation of the protein of interest within the cells. The fermentation profile for one of the example (pLMAB-hGH) grown in the 1 litre fermenter is shown in the FIG. 6 The protein accumulation was measured by scanning Coomassie stained SDS-PAGE gels of whole cell lysates by the standard method. As is evident from Table 3, pLMAB-LacZ#12 yielded three fold as compared to pBHL which is a commercial vector at the end of 6 hours from induction. There is a linear relationship between time of induction and expression and the surprising effect is 1.15 fold higher protein expression levels at the end of 22 hour fermentation run after induction as compared to the commercial pBHL vector. The most surprising effect is consistent expression levels achieved after just six hours after induction till the end of fermentation for 22 hours after induction with pLMAB vector.

The experimental details and results obtained are tabulated in Table 4 below.

TABLE 4
Percent expression of Total protein (TP) of the samples as seen on SDS-
PAGE of different proteins using pLMAB Expression vector (which has
single gene insert) during the fermentation process described above.

| | | Hours | | % expression |
|---|---|---|---|---|
| | w/w Feed | of | | of the |
| | composition | fermen- | | protein of |
| Clone | (Glucose:YE) | tation | OD_{600 nm} | interest |
| pLMAB-rhGH #16* | 20:15 | 22 | 54.8 | 24.89 |
| pLMAB-EK #6 | 20:15 | 20 | 51.8 | 26.29 |
| pLMAB-rhIL2 #4* | 20:20 | 20.5 | 49.6 | 27.43 |
| pLMAB-rhRete #3* | 25:20 | 22 | 49.8 | 9.3 |
| pLMAB-OmpC #1* | 20:20 | 22 | 44 | 9.06 |

*#16/4/3/1 signifies selected high expressing clones respectively.

### Example 13

Plasmid copy number of pRA LACZ, pBAD-His-LacZ and pLMAB was checked at an interval of one year as shown in Table 5 below.

TABLE 5
Analysis of Plasmid Copy number

| Name of | Replica | | | | |
|---|---|---|---|---|---|
| Vector | 1 | 2 | 3 | 4 | Average ± SD |
| pRA LACZ | — | — | 183 | 174 | 178 ± 4 |
| pBAD-His- | — | — | 167 | 200 | 183 ± 16 |
| LacZ | | | | | |
| pLMAB | 82 | 123 | 90 | 111 | 101 ± 16 |

While the present invention is described above in connection with preferred or illustrative embodiments, these embodiments are not intended to be exhaustive or limiting of the invention. Rather, the invention is intended to cover all alternatives, modifications and equivalents included within its spirit and scope, of the invention.

We claim:

1) An expression vehicle comprising an isolated nucleic acid as shown in Seq ID No. 1 comprising of a synthetic hybrid promoter wherein the hybrid promoter comprises of an inducible arabinose promoter derived from E. coli and a synthetic stretch of 8-35 nucleotides from pHO regulon introduced in the region between −55 to −10 and ribosome binding site from the transcription initiation site.

2) The isolated nucleic acid of claim 1, wherein the inducible arabinose promoter sequence consists of a stretch of 17-32 nucleotides introduced in the region between −55 to −10 and ribosome binding site from the transcription initiation site selected from the group consisting of Seq ID No. 15, Seq ID No. 16, Seq ID No. 17, Seq ID No. 18, Seq ID No. 19, Seq ID No. 20, Seq ID No. 21 and Seq ID No. 22.

3) The isolated nucleic acid of claim 2, wherein the inducible arabinose promoter sequence consists of a stretch of 17 nucleotides as shown in Seq. ID No. 15, introduced in the region between −55 to −10 and ribosome binding site from the transcription initiation site.

4) An expression vector comprising a synthetic hybrid promoter as shown in Seq ID No. 1.

5) An expression vector of claim 4, wherein the inducible arabinose promoter sequence consists of a stretch of 17 nucleotides as shown in Seq. ID No. 15, introduced in the region between −10 and ribosome binding site from the transcription initiation site operably linked to heterologous protein characterized in that the expression is induced after 3 hours of induction.

6) An expression vector of claim 4, wherein the inducible arabinose promoter sequence consists of a stretch of 17 nucleotides as shown in Seq. ID No. 15, introduced in the region between −10 and ribosome binding site from the transcription initiation site operably linked to heterologous protein wherein at least 100 fold protein expression is obtained as compared to the yield obtained under uninduced conditions.

7) A prokaryotic host cell harboring the expression vehicle comprising an isolated nucleic acid as shown in Seq ID No. 1.

8) The use of prokaryotic host cell of claim 7 for the increased expression of heterologous genes coding for polypeptides.

9) A prokaryotic host cell harboring the expression vehicle comprising an isolated nucleic acid as shown in Seq ID No. 1 with an ATCC accession no. PTA-9371.

10) The use of prokaryotic host cell of claim 9 for the increased expression of heterologous genes coding for polypeptides.

11) A prokaryotic host cell harboring a high plasmid copy number expression vehicle comprising an isolated nucleic acid as shown in Seq ID No. 1 with an ATCC accession no. PTA-9371.

12) The expression vehicle comprising an isolated nucleic acid as shown in Seq ID No. 1 as substantially described herein with respect to the foregoing examples 1 to 13 and drawings 1 to 7.


SEQUENCE LISTING (14 sequences)

SEQ ID NO: 1 - DNA, 4156 bp, ARTIFICIAL
  completely synthesized
aagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgctttcatatctttcactttttttgggctaacaggaggaattaaccatggcactgcacgcacatctggaccctcatctggtgacggagcacgcccacctcgatccgcacgctagcctgcacgcacatctggaccctcatctggtgacggagcacgcccacctcgatccgcacgtcgacgctagcgacgacgacgacaaggtacctaggctcgagatctgtacactagtataagcttggctgttttggcggatgagagaagattttcagcctgatacagattaaatcagaacgcagaagcggtctgataaaacagaatttgcctggcggcagtagcgcggtggtccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatacgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 2 - DNA, 1102 bp, ARTIFICIAL
  completely synthesized
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgggcggtgcgcatcatcatcatcatcacgtcgacgacgacgacgacaaggatcgttggggatccgagctcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatacgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggta

SEQ ID NO: 3 - DNA, 32 bp, ARTIFICIAL
  SYNTHETIC PRIMER
aaatcaagtcgcccatcaagcagccaataatc

SEQ ID NO: 4 - DNA, 30 bp, ARTIFICIAL
  SYNTHETIC PRIMER
tgaaaggttatattctcaatctcaccattc

SEQ ID NO: 5 - DNA, 29 bp, ARTIFICIAL
  SYNTHETIC PRIMER
ccgtattggcaaatattgacggccagtta

SEQ ID NO: 6 - DNA, 25 bp, ARTIFICIAL
  SYNTHETIC PRIMER
gccaaaatcgaggccaattgcaatc

SEQ ID NO: 7 - DNA, 34 bp, ARTIFICIAL
  SYNTHETIC PRIMER
atccctgcatgccaggcgtgccagaaacttaact

SEQ ID NO: 8 - DNA, 34 bp, ARTIFICIAL
  SYNTHETIC PRIMER
cgaggccaattgcaatcgccatggtttcactcca

SEQ ID NO: 9 - DNA, 31 bp, ARTIFICIAL
  SYNTHETIC PRIMER
cttttgatactcccgccattcagagaagaaa

SEQ ID NO: 10 - DNA, 31 bp, ARTIFICIAL
  SYNTHETIC PRIMER
atatgaaagcgggtatggagaaacagtagag

SEQ ID NO: 11 - DNA, 32 bp, ARTIFICIAL
  SYNTHETIC PRIMER
tctttcactttttttgggctaacaggaggaat

SEQ ID NO: 12 - DNA, 32 bp, ARTIFICIAL
  SYNTHETIC PRIMER
gtagggtctcatgagcgtatacatatttgaat

SEQ ID NO: 13 - DNA, 58 bp, ARTIFICIAL
  SYNTHETIC PRIMER
gaccgaaaacctgtattttcgtcgacgacgacgacaaggctgaaatttataataaaga

SEQ ID NO: 14 - DNA, 30 bp, ARTIFICIAL
  SYNTHETIC PRIMER
gttttatcagaccgcttctgcgttctgatt